(19) United States
(12) Patent Application Publication
(10) Pub. No.: US 2026/0335375 A1
(43) Pub. Date: Oct. 29, 2026

(54) SYNTHETIC AMNIOTIC FLUID COMPOSITIONS AND MEHTODS OF USING SAME

(71) Applicants: CHILDREN'S HOSPITAL MEDICAL CENTER, Cincinnati, OH (US); UNVERSITY OF CINCINNATI, Cincinnati, OH (US)

(72) Inventors: MARC ORIA, Cincinnati, OH (US); BRAXTON FORDE, Cincinnati, OH (US); JOSE LUIS PEIRO-ILBANEZ, Cincinnati, OH (US)

(21) Appl. No.: 19/122,315

(22) PCT Filed: Oct. 24, 2023
(86) PCT No.: PCT/US2023/035776

§ 371 Date: Apr. 18, 2025

Related U.S. Application Data
(60) Provisional application No. 63/418,751, filed on Oct. 24, 2022.

(51) Int. Cl. A61K 35/50; A61K 31/191; A61K 31/375; A61K 31/7004; A61K 38/38; A61K 38/57; A61P 15/06; C12N 5/073

(52) U.S. Cl. CPC A61K 35/50; A61K 31/191; A61K 31/375; A61K 31/7004; A61K 38/38; A61K 38/57; A61P 15/06; C12N 5/0605

(57) ABSTRACT

Disclosed are synthetic amniotic fluid compositions comprising ulin-A-statin/urinary trypsin inhibitor (UTI) and ascorbic acid and methods of using the disclosed synthetic amniotic fluid compositions, which may be useful for a variety of purposes related to treatment of a fetus, replacement of amniotic fluid, or research uses in which amniotic cells or tissues are used. The synthetic amniotic fluid compositions may be used to minimize and/or reduce risks associated with currently utilized compositions for amnioinfusion, which traditionally include normal saline (NS) and Lactated Ringer's (LR), or to improve amniotic epithelial cell viability and integrity, in vivo and/or in vitro.

### CROSS-REFERENCE TO RELATED APPLICATIONS

This application claims priority to and benefit of U.S. Provisional Application No. 63/418,751, filed Oct. 24, 2022, the contents of which are incorporated in their entirety for all purposes.

### BACKGROUND

Since the first case in 1981, the field of fetal surgery has continued to expand. However, the greatest limitation to more fetal interventions being offered prenatally is the high complication rate, which can range from as low as one in several hundreds to as high as 96% depending on the procedure. The most pressing complication from fetal interventions is preterm birth. A primary driver of preterm birth is preterm pre-labor rupture of membranes (PPROM) which typically leads to preterm labor and birth. The reason PPROM rates can be high after surgery is that the amniotic membrane does not heal after surgery, therefore if a defect is created into the amniotic sac (as occurs with any prenatal intervention), the surgeon must rely on the strength of the chorion-amnion seal (which occurs naturally at 15-16 weeks and remains for the duration of the pregnancy), to prevent the rest of the amniotic sac from experiencing ruptured membranes. Numerous attempts have been made to patch or plug the surgical defect created in the amniotic membrane, rarely providing benefit, but also sometimes causing harm. At the time of in-utero fetal interventions, amnioinfusions (infusion of an outside fluid into the amniotic cavity) are typically required, either as part of the therapy being performed or to optimize the surgical view. The fluid used is Normal Saline (NS) or most frequently, Lactated Ringer's (LR), both of which are significantly more nutrient poor and acidic than normal amniotic fluid.

Thus, currently available compositions for use with amnion tissue, such as in the case of amnioinfusions during in-utero fetal interventions, are suboptimal, and further improvements are needed. The instant disclosure seeks to address one or more of the aforementioned needs in the art.

### BRIEF SUMMARY

Disclosed are synthetic amniotic fluid compositions comprising one or both of ulin-A-statin/urinary trypsin inhibitor (UTI) and ascorbic acid and methods of using the disclosed synthetic amniotic fluid compositions, which may be useful for a variety of purposes related to treatment of a fetus, replacement of amniotic fluid, or research uses in which amniotic cells or tissues are used. The synthetic amniotic fluid compositions may be used to minimize and/or reduce risks associated with currently utilized compositions for amnioinfusion, which traditionally include normal saline (NS) and Lactated Ringer's (LR), or to improve amniotic epithelial cell viability and integrity, in vivo and/or in vitro.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Apoptosis in human amniotic epithelium after simulated amnioinfusion. ApoTracker Green and Viability Dye eFluor 780 result immediately at conclusion of the simulated amnioinfusion (day 5 of culture, row 1) and after an additional 48 hours to allow to see if cells would recover if left in culture for an additional 48 hours post infusion (day 8 of culture, row 2). Column 1 is control, column 2 is NS, column 3 is LR, column 4 is AL. Q1 are 780+/AG-consistent with necrotic cells, Q2 are 780+/AG+consistent with cell death via apoptosis, Q3 are 78−−/AG+consistent with living but early apoptotic cells, and Q1 are 780−AG/−consistent with living cells. At conclusion of the simulated amnioinfusion, 85% of cells in control were living without signs of early apoptosis, compared with 44% in NS, 53% in LR, and 89% in AL. After cells were allowed to recover, 88% of cells in control were living without signs of early apoptosis, compared with 21% after previous NS exposure.

FIG. 2. Light microscopy revealed multiple signs of cell death occurring in tandem with cellular shrinkage, nuclear fading, and membrane blebbing. Images taken at 20×. Top Left-Control, Top Right-Normal Saline, Bottom Right-Amnio-well, Bottom Left-Lactated Ringer's.

FIG. 3. A. Relative ROS expression between groups as measured by fluorescent intensity. B. Immunofluorescent imaging showing rescue of ROS production with ascorbic acid and UTI. ROS=reactive oxygen species; UTI=urinary trypsin inhibitor.

FIG. 4. Relative P21, Caspase 3, and BCL2 to BAX ratio after simulated amnioinfusion with various formulations of Amnio-well. Amnio-well+UTI was most similar to control in the apoptotic pathways and had the highest BCL2 to BAX ratios (with a low BCL2 to BAX ratio and high BAX being implicated in ROS-mediated cell death pathways). ROS=reactive oxygen species; UTI=urinary trypsin inhibitor.

FIG. 5 depicts mRNA levels of p21 in response to various levels of vitamin C, urinary trypsin inhibitor, control, and normal saline (NS).

FIG. 6 depicts mRNA levels of GSTP1 in response to various levels of vitamin C, urinary trypsin inhibitor, control, and normal saline (NS).

FIG. 7 depicts mRNA levels of BAX in response to various levels of vitamin C, urinary trypsin inhibitor, control, and normal saline (NS).

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the synthetic amniotic fluid compositions and/or methods as described herein, as well as any additional or optional element described herein or otherwise useful in the manufacture or use of synthetic amniotic fluid compositions.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” may be used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

“Substantially free” with respect to a component, class of components or combinations of components that has or have been specifically identified herein, means no effective amount of that specifically identified component, class of components or combinations of components, or from about 1 wt. % or less, from about 0.1 wt. % or less, or even from about 0.01 wt. % or less, or 0% (i.e., completely free) of the specifically identified component or class of components as specified herein. In one aspect, “substantially free” means that the ingredient is not intentionally added.

### Amniotic Fluid Compositions

The present disclosure relates to synthetic amniotic fluid compositions, and methods of using the disclosed synthetic amniotic fluid compositions. The synthetic amniotic fluid compositions disclosed herein may be useful for a variety of purposes related to treatment of a fetus, replacement of amniotic fluid, or research uses in which amniotic tissues are used. The synthetic amniotic fluid compositions may be used to minimize and/or reduce risks associated with currently utilized compositions for amnioinfusion, which traditionally include normal saline (NS) and Lactated Ringer's (LR). Such currently used substitute fluids have increased acidity and are nutrient poor, and further, may cause widespread damage to the amnion and may accelerate amniotic epithelial cell death, particularly during fetal surgery procedures. Consequently, existing compositions used for amnioinfusion may increase the risk of membrane breakdown, premature preterm premature rupture of membranes (PPROM), and preterm birth.

Conventional solutions such as NS and LR, for instance, have been demonstrated to trigger cell damage and death in human amniotic epithelium. Disclosed are alternate synthetic amniotic compositions that may be used in lieu of NS and LR, and which avoid some or all of the disadvantages of currently used substitute compositions, and which have been found to have unexpected, synergistic benefits as compared to existing compositions.

Specifically, the disclosed novel synthetic amniotic fluid compositions comprise vitamin C (ascorbic acid) and UTI in amounts that yield an unexpected and synergistic outcome. While the level of vitamin C in naturally occurring amniotic fluid typically falls within the range of about 91 mg/L to 574 mg/L, and normal UTI in pregnancy is from about 14.5 mg/L to about 37.5 mg/L, Applicant has found that, at higher concentrations of vitamin C, further in combination with the disclosed amounts of UTI, the disclosed synthetic amniotic fluid compositions may be contacted with amniotic epithelial cells without the degree of cell damage and death observed with NS and LR, based on in vitro cell data.

Thus, the disclosed synthetic amniotic fluids may be useful in a variety of contexts, ranging from serving as substitutes for natural amniotic fluid to facilitating the delivery of therapeutic agents to a developing fetus in vivo via the amnion.

In one aspect, the disclosed synthetic amniotic fluid composition may comprise ulin-A-statin/urinary trypsin inhibitor (UTI). Urinary trypsin inhibitor (UTI, also known as ulinastatin, mingin, human inhibitor 30, serpin, miraclid, urinastatin (in Japanese literature) and bikunin) is a multivalent serine protease inhibitor synthesized and released in human urine and blood. UTI is an acidic glycoprotein, composed of 143-amino acid residues. www.biovendor.com/urinary-trypsin-inhibitor, Takano, H., Inoue, Ki., Shimada, A. et al. Urinary trypsin inhibitor protects against liver injury and coagulation pathway dysregulation induced by lipopolysaccharide/D-galactosamine in mice. Lab Invest 89, 833-839 (2009). doi.org/10.1038/labinvest.2009.35. UTI is secreted when inter-α-trypsin inhibitors are degraded by neutrophilic elastase. UTI has many physiologic effects, including the inhibition of neutrophilic elastase, trypsin, α-chymotrypsin, plasmin, and cathepsin G. Han, Jong In. “Urinary trypsin inhibitor: miraculous medicine in many surgical situations,” Korean Journal of Anesthesiology vol. 58, 4 (2010): 325-7. doi: 10.4097/kjae.2010.58.4.325. UTI is commercially available from Bio Vendor R&DR.

In one aspect, the disclosed synthetic amniotic fluid composition may comprise ascorbic acid. Vitamin C is a water-soluble vitamin, antioxidant, and essential co-factor for collagen biosynthesis, carnitine and catecholamine metabolism, and dietary iron absorption. Humans are unable to synthesize vitamin C, such that it must be obtained through the dietary intake of fruits and vegetables. Abdullah M, Jamil R T, Attia F N. Vitamin C (Ascorbic Acid) StatPearls Publishing; 2023 January. Available from: www.ncbi.nlm.nih.gov/books/NBK499877/.

The disclosed synthetic amniotic fluid compositions may comprise both UTI and ascorbic acid. The UTI and ascorbic acid may be present in the synthetic amniotic fluid composition in a predetermined ratio. For example, the synthetic amniotic fluid composition may comprise UTI and ascorbic acid at a ratio of about 1:10, or about 0.5 to about 10 or about 0.6 to about 10, or about 0.7 to about 10, or about 0.8 to about 10, or about 0.9 to about 10, or about 1.1 to about 10, or about 1.2 to about 10, or about 1.3 to about 10.

In one aspect, the amount of UTI in the synthetic amniotic fluid composition may be, for example, from about 10 μg/mL to about 500 μg/mL UTI, or about 15 μg/mL to about 400 μg/mL UTI, or about 20 μg/mL to about 300 μg/mL UTI, or about 25 μg/mL to about 200 μg/mL UTI, or about 185 μg/mL UTI of the composition. In one aspect, the amount of ascorbic acid in the synthetic amniotic fluid composition may be, for example, from about 400 μg/mL to about 2000 μg/mL ascorbic acid, or about 500 μg/mL to about 1500 μg/mL ascorbic acid, or about 550 μg/mL to about 1000 μg/mL ascorbic acid, or about 600 μg/mL to about 800 μg/mL ascorbic acid.

The disclosed synthetic amniotic fluid compositions may comprise ascorbic acid in any of the aforementioned amounts in combination with UTI in any of the aforementioned amounts. For example, in one aspect, the synthetic amniotic fluid composition may comprise from about 37.5 mg/L to about 75 mg/L UTI and from about 400 mg/L to about 800 mg/L ascorbic acid.

The synthetic amniotic fluid composition may further comprise additional components, for example, sodium chloride, sodium gluconate, albumin, potassium chloride, d-glucose, magnesium chloride, zinc chloride. In one aspect, the synthetic amniotic fluid composition may comprise about 135 mEq/L sodium. In one aspect, the synthetic amniotic fluid composition may comprise about 105 mEq/L chloride. In one aspect, the synthetic amniotic fluid composition may comprise about 4.5 mmol/L potassium. In one aspect, the synthetic amniotic fluid composition may comprise about 6 mg/dL calcium. In one aspect, the synthetic amniotic fluid composition may comprise about 2 mg/dL magnesium. In one aspect, the synthetic amniotic fluid composition may comprise about 0.3 mg/dL albumin. In one aspect, the synthetic amniotic fluid composition may comprise 30 mg/dL glucose.

In one aspect, the synthetic amniotic fluid composition may further comprise a nutrient, an amino acid, a fatty acid, a hormone, a growth factor, and combinations thereof. The synthetic amniotic fluid composition may have a physiological pH, for example, a pH of about 7.4.

The disclosed synthetic amniotic fluid compositions are distinct from naturally occurring amniotic fluid, in that the synthetic amniotic fluid compositions lack at least one component found in naturally occurring amniotic fluid and/or comprise at least one component that is not found in naturally occurring amniotic fluid. For example, in one aspect, the synthetic amniotic fluid composition is substantially free of interleukin 6 (IL6). In one aspect, the synthetic amniotic fluid composition is substantially free of cytokines. In one aspect, the synthetic amniotic fluid composition is substantially cell-free. In one aspect, the synthetic amniotic fluid composition is substantially free of meconium products. In one aspect, the synthetic amniotic fluid is sterile. In one aspect, the synthetic amniotic fluid composition may be at a temperature appropriate for storage, and/or delivery of the synthetic amniotic fluid composition, for example, the synthetic amniotic fluid composition may be at a temperature of −32 °C for storage, or at a temperate of less than −32 °C, for example −80 °C for long term storage. In other aspects, the synthetic amniotic fluid composition may have a temperature of approximately 37 °C, for example when administering to an individual or the amniotic sac of an individual.

In other aspects, the disclosed synthetic amniotic fluid compositions may be used to contain a therapeutic agent, for example, for delivery to a fetus in utero. Therapeutic agents may include, for example, an antibiotic, a biologic, a small molecule therapeutic, a vitamin, a nutrient, and combinations thereof. The synthetic amniotic fluid compositions may comprise less than 5% of a therapeutic agent, or less than 4%, or less than 3%, or less than 2%, or less than 1% or less than 0.5%, or less than 0.4%, or less than 0.3%, or less than 0.2%, or less than 0.1%, or less than 0.05%, or less than 0.04%, or less than 0.0.3%, or less than 0.02%, or less than 0.01% of a therapeutic agent.

The disclosed synthetic amniotic fluid compositions may be used for a variety of purposes, including therapeutic or research purposes, both in vitro and in vivo.

In one aspect, disclosed is a method for reducing the likelihood of a premature delivery, comprising administering a synthetic amniotic fluid composition as disclosed herein, to an individual in need thereof. In one aspect, the individual may be one at risk of postoperative PPROM. In one aspect, the individual has undergone fetoscopic myelomeningocele (MMC) closure. In one aspect, the individual has undergone open MMC closure.

The method may include introducing a synthetic amniotic fluid composition as disclosed herein into the amniotic sac during pregnancy. In one aspect, the individual being treated is the pregnant individual, who has, or is likely to have, low amniotic fluid levels (oligohydramnios). Detection of amniotic fluid levels may be determined via ultrasound or other diagnostic tests, or low amniotic fluid levels may be anticipated where a surgical procedure is to be carried out on the individual. The synthetic amniotic fluid composition may be administered via a sterile catheter or tube inserted into the amniotic sac. In one aspect, this is carried out via ultrasound guidance, and using a controlled rate of infusion to prevent excessive pressure on the fetus and amniotic sac. The amount of fluid may vary. The synthetic amniotic fluid composition may be administered via transabdominal infusion and/or amnioinfusion. The administering may be carried out over a period of time and for the number of times sufficient to achieve the desired therapeutic effect. For example, the composition may be administered via an amnioinfusion at least one time per week, at least two times per week, at least three times per week, at least four times per week, at least five times per week, or at least six times per week, or daily. In one aspect, the synthetic amniotic fluid composition is administered during a fetal intervention.

In one aspect, disclosed is a method for improving viability of an amnion epithelial cell, comprising contacting said amniotic epithelial cell with a synthetic amniotic fluid composition as disclosed herein. The contacting may be for a time and duration sufficient to achieve the desired effect, for example, for a period of time of from one hour to two weeks, or from two hours to one week, or from three hours to six days, or from four hours to five days, or from five hours to four days, or from six hours to three days, or from seven hours to two days, or from eight hours to 24 hours. In one aspect, the improvement is characterized by reduced cellular shrinkage. In one aspect, the improvement is characterized by reduced nuclear fading. In one aspect, the improvement is characterized by reduced membrane blebbing. The contacting may be via any number of methods, for example, via amnioinfusion or in cell culture. In one aspect, the epithelial cell may be from, or comprise an amniotic tissue. The contacting may be ex vivo and/or in vivo. The epithelial cell may be a mammalian epithelial cell, or mor particularly, a human epithelial cell obtained from the amnion or amniotic sac.

In one aspect, disclosed is a method of replacing or supplementing a portion of an amniotic fluid in an individual, comprising administering a synthetic amniotic fluid composition as disclosed herein. In further aspects, disclosed is a method of restoring and/or improving amniotic fluid volume in an individual in need thereof, comprising administering a synthetic amniotic fluid composition as disclosed herein to the amniotic sac of said individual. In one aspect, disclosed is a method of administering a therapeutic agent to the fetus, comprising administering a synthetic amniotic fluid composition as disclosed herein, the synthetic amniotic fluid composition comprising at least one therapeutic agent as described herein.

### EXAMPLES

The following non-limiting examples are provided to further illustrate aspects of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific aspects that are disclosed and still obtain a like or similar result without departing from the spirit and scope of the invention.

### Example 1. Creation of a Novel Synthetic Amniotic Fluid

Objective: Fluid used for amnioinfusion during fetal interventions (Normal Saline (“NS”) and Lactated Ringer's (“LR”)) is significantly more acidic and nutrient poor than typical amniotic fluid. Disclosed are synthetic amniotic fluid compositions and its impact on human amniotic epithelium.

STUDY DESIGN: Amniotic epithelial cells from term placentas were isolated and cultured. A synthetic amniotic fluid was created with similar electrolyte, pH, albumin, and glucose concentrations to human amniotic fluid, termed “Amnio-well,” (“AL”). The cultured human amniotic epithelium was exposed to normal saline solution, lactated Ringer's solution, and Amnio-well. As a control, one group of cells remained in culture media. Cells were evaluated for apoptosis and necrosis. A second analysis to examine if cells could be “rescued” was performed, wherein the cells were allowed to remain in the culture media for an additional 48 hours after amnioinfusion. Subsequently, tissue testing with human amniotic membrane explants was evaluated similarly. Immunofluorescent intensity studies were undertaken to evaluate reactive oxygen species-mediated cell damage. Real-time quantitative polymerase chain reaction was used to evaluate gene expression in apoptotic pathways.

Results: With simulated amnioinfusion, 44%, 52%, and 89% of amniotic epithelial cells were alive after exposure to normal saline solution, lactated Ringer's solution, and Amnio-well, respectively, compared with 85% in control (P<0.001). After amnio infusion and attempted cell rescue, 21%, 44%, 94%, and 88% of cells were alive after exposure to normal saline solution, lactated Ringer's solution, Amnio-well, and control, respectively (P<0.001). In simulated amnioinfusion with full-thickness tissue explants, 68%, 80%, 93%, and 96% of cells were viable in normal saline solution, lactated Ringer's solution, Amnio-well, and control, respectively (P<0.001). In culture, reactive oxygen species production was higher in normal saline solution, lactated Ringer's solution, and Amnio-well than in control (4.9-, 6.6-, and 1.8-fold higher, respectively, P<0.001); however, this could be mitigated in Amnio-well by adding ulin-A-statin and ascorbic acid. Gene expression data revealed abnormal signaling in the p21 and BCL2/BAX pathways with normal saline solution compared with control (P=0.006 and P=0.041); changes were not seen with Amnio-well.

Conclusion: In vitro, normal saline and lactated Ringer's solutions caused increased amniotic membrane reactive oxygen species and cell death. The use of a novel fluid similar to human amniotic fluid led to the normalization of cellular signaling and less cell death.

### Materials and Methods Human Amnion Tissue and Cell Isolation

Under compliance with Cincinnati Children's Hospital Medical Center (IBC 20190071, IBC 2022-0098), human amnions were isolated from term placentas of deidentified patients that underwent cesarean delivery from 2020 to 2022. All deliveries which were positive for infectious agents, including HBV, HCV, and HIV and those with pre-diagnosed genetic abnormalities were excluded from this study. All the materials and equipment used were sterile.

### Culture and Amnioinfusion Model

Amnions were digested and cells isolated per previously published protocols. Once a successful in vitro model human amniotic epithelial cells was established, cells were grown in standard cell culture media of DMEM/F12 (Gibco, Thermo Fisher Scientific) supplemented with 10% FBS, 10 U/mL penicillin, 100 ug/mL streptomycin (1×, Sigma Aldrich, St. Louis, Missouri) and 10 ng/mL epidermal growth factor (Thermo Fisher Scientific, Waltham, Massachusetts). Human amniotic epithelial cells were plated in six well plates and grown in standard culture media until at least 80% confluence of cellular growth was reached. Cells were exposed to NS, LR, or AL (Table), with control cells undergoing exchange of fresh culture media. Because amniotic fluid can experience turnover as quickly as 24 hours, the normal amniotic fluid dynamics were simulated. To do this, at 6, 12, 18, and 24 hours, 25%, 50%, 75%, and 100%, respectively of the exposure fluid was replaced with fresh culture media. Control cells continued to only undergo exchange with fresh culture media. All fluid was warmed to 37 °C.

### Flow Cytometry

After isolation, cells were checked for purity. Briefly, cells dissociated with TrypLE for 10 minutes then washed with the medium and washed once more with FACS buffer. Cells were stained with different antibodies at room temperature for 15 minutes. Afterward, the cells were washed 3 times using cell staining buffer (Biolegend, San Diego, California) and centrifuged. Intracellular staining with FITC-conjugated-anti-cytokeratin (Biolegend, San Diego, California) was performed after fixation and cells permeabilization according to the manufacturer's instructions (eBioscience, San Diego, California). Specific antibodies selected were as per previously published protocols. Markers used were as follows: for mesenchyme cells (CD14−, CD45−, CD90+, and CD105+) epithelial (CD14−, CD45−, CD90−, EpCam+, and PanCK+) and immune or hematopoietic (CD14+, CD45+, CD90−, EpCam−, and PanCK−) and analyzed by flow cytometry on an LSRII (BD Pharmingen).

Apotracker Green and Viability Dye eFluor 780

After 24 hours of simulated amnioinfusion, cells were permeabilized, fixed, stained, and evaluated via flow cytometry for the number of live cells, early apoptotic cells, late apoptotic cells, and necrotic cells using fixable Viability Dye eFluor 78013 and Apotracker Green (Apotracker Green, Biolegend Inc, San Diego, California). Apotracker Green is a calcium-independent probe for detecting apoptotic cells by detecting translocation of phosphatidylserine residues to the cell surface in a cell undergoing apoptosis. This probe is used in conjunction with a dead cell indicator to identify living and dead cells as well as apoptotic and nonapoptotic cells. Cells staining for live/nonapoptotic are living, live/apoptotic are in early apoptosis, dead/apoptotic are in late apoptosis, and dead/nonapoptotic are necrotic. Viability Dye eFluor 780 is a cell-impermeant, amine-reactive dye, which only penetrates the cell membrane in dead cells, binding to internal proteins, resulting in bright fluorescence. The percentage of cells labeled as 780+AG+ (late apoptosis), 780+AG-(necrosis), 780-AG+ (early apoptosis), and 780-AG-(live cells) was quantified and analyzed (FlowJo, BD Life Science). Cells were also imaged via light microscopy every 6 hours during the amnioinfusion to evaluate the subjective cellular changes occurring.

To evaluate if ongoing cell death occurs or if cells could be “rescued” by reintroduction to culture media, the experiment was repeated; however, after the complete replacement of the exposure fluid by culture media, the cells were left in culture media for an additional 48 hours to evaluate if ongoing cell death was present.

### Organo-Type Tissue Explant Testing

After this confirmation, immediately after isolation of human amnion from the placenta, <3-mm full-thickness sections of amniotic membranes were isolated and exposed to the various fluids in the same manner as described above. Live/dead staining was then performed and analyzed via immunofluorescence, with cell counting for live/dead staining done with ImageJ software (ImageJ, NIH.gov, Bethesda, Maryland).

### Oxidative Stress Analysis

Reactive oxygen species (ROS) have been strongly implicated in the pathogenesis of PPROM; thus, to further evaluate the cause of cell death, cells were evaluated for the production of ROS after the simulated amnioinfusions. During this experimentation, various formulations of AL with added antioxidants were tested. Due to the implication of ascorbic acid deficiency and PPROM, ascorbic acid was diluted into AL at physiologic concentrations. Another antioxidant and anti-inflammatory found in healthy amniotic fluid, ulin-A-statin/urinary trypsin inhibitor (UTI) which has been implicated in preterm labor was added at physiologic concentrations (Table).

Levels of ROS were determined by using the cellular ROS assay kit (ab113851/Abcam DCFDA assay, Abcam, Cambridge, United Kingdom), following the manufacturer's instructions. Briefly, cells were plated on 96-well black-walled plates at 25,000 cells per well and monitored until cells reached >80% confluence. Cells were then washed and stained with 25-mM 2′-7′ dichlorofluorescin diacetate (DCFH-DA) for 45 minutes at 37 °C in the dark. Subsequently, cells were washed and exposed to NS, LR, AL, or AL with UTI and vitamin C. Controls underwent exchange with culture media without phenol red. After 6 hours of incubation (per the manufacturer's protocol), DCFH-DA was detected by fluorescence spectroscopy, with excitation/emission at 485/535 nm in a microplate reader. Cells without stain were run as negative controls. This experiment was repeated with cells on coverslips and cells were imaged through FITC fluorescent lens per the manufacturer's protocol.

### RNA Extraction and Reverse Transcription-Quantitative Polymerase Chain Reaction (RT-qPCR) Analysis

Cells in culture were lysed in RLT buffer, and RNA was extracted using an RNeasy Plus Mini Kit (Qiagen Science, Hilden, Germany) following the manufacturer's protocol. Samples were quantified by spectrophotometry (Epoch Biotek, Biotek Instruments, Winooski, Vermont). A 1-mg RNA/sample was reverse transcribed into cDNA using the RT2 First Strand Kit (Qiagen Sciences, Germantown, MD). A 1-mg cDNA sample was used to set up Reverse transcription-quantitative polymerase chain reaction (RT-qPCR) using TaqManR gene expression assay (Applied Biosystems, Foster City, CA) and 7500 Fast Real-Time PCR System. Target genes were normalized using GAPDH as endogenous control; their relative quantification of transcript expression was performed using the 2-D DCt method (Ct represents the threshold cycle). Samples were run in duplicate.

### Statistical Analysis

All datasets were analyzed for normalcy and homoscedasticity. Data were normally distributed. Data were analyzed by 1-way ANOVA for continuous variables and chi-squared for categorical data. Analyses were performed with GraphPad Prism version 9.0 (GraphPad Software). A P value <0.05 was considered statistically significant.

### Results

### High Fidelity of Culture

Flow cytometry revealed high purity of isolation, at or better than reported in the literature with >99% of cells expressing PanCK and 90% to 95% of cells expressing EPCAM. Less than 1% of cells expressed hematopoietic or immune markers, and <5% of cells expressed mesenchyme markers. Flow cytometry was repeated at D9 of culture and continued high purity was confirmed with >99% PanCK expression, >95% EPCAM expression, <1% hematopoietic or immune markers, <2% CD105 expression (mesenchyme), and <10% CD90 expression (mesenchyme) (data not shown).

### Amnio-Well Amnioinfusion Protected Against Apoptosis in Cell Culture.

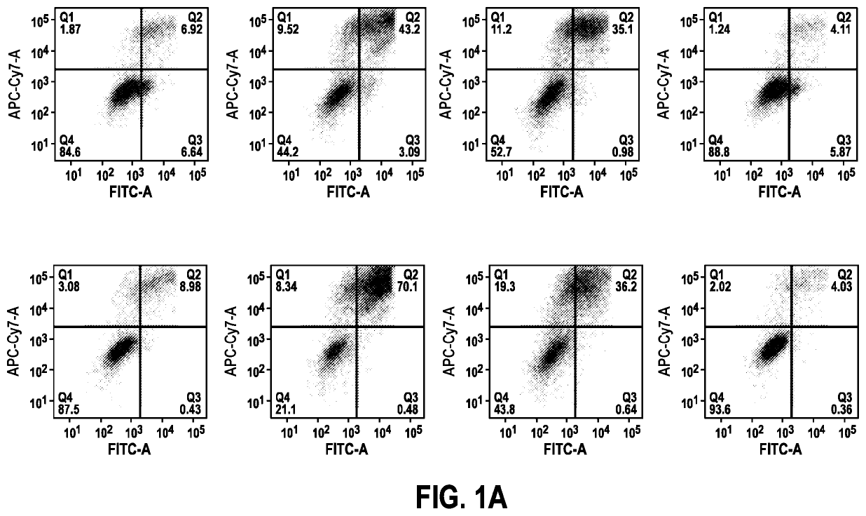

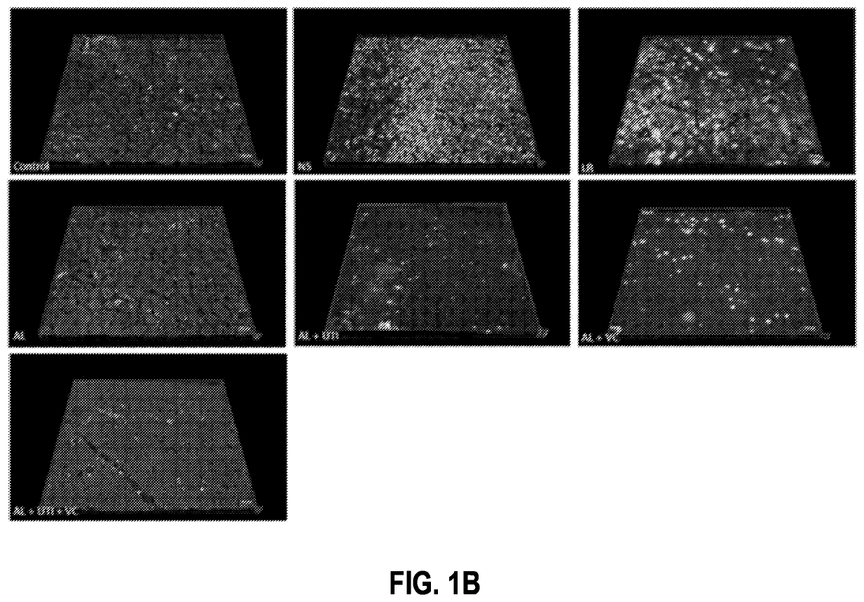

To explore the effect of amnioinfusion, cells underwent simulated amnioinfusion as described above and staining for viability and apoptosis. When comparing results after the initial 24 hours of simulated amnioinfusion, only 44% and 52% of cells exposed to NS and LR, respectively were still alive, compared with 85% for control and 89% in AL (P<0.001) (FIG. 1). After repeat experimentation, with attempted rescue by leaving cells in culture media for 48 hours after the experiment, flow cytometry revealed that of the cells exposed to NS and LR, significantly more cell death occurred, now with only 21% and 44% of cells still alive between, compared to 88% of control and 94% exposed to AL (P<0.001) (FIG. 1). Light microscopy revealed multiple signs of cell death occurring in tandem with cellular shrinkage, nuclear fading, and membrane blebbing (FIG. 2).

Decreased Cell Death with Amnio-Well in Tissue Explants.

Small segments (<3 mm) of full-thickness amnion were isolated from the previously described placentas. Live/dead staining of these sections of amnion was performed daily, using the LIVE/DEAD Viability/Cytotoxicity for mammalian cells assay (Thermo Fisher, XX) and we confirmed that amnion sections could be preserved for up to 4 to 5 days in culture media before significant degradation occurred. Given this, at the time of placental collection, small portions of full-thickness amnion were isolated and underwent simulated amnioinfusion with NS, LR, AL, and culture media as previously described. After the simulated amnioinfusion, cells exposed to NS and LR had 32% and 20% of cells stain as dead/nonviable, respectively, vs only 7% of cells in AL and 4% of control (P<0.001).

Up-regulation of reactive oxygen species production with normal saline and lactated Ringer's and negation of reactive oxygen species with Amnio-well+antioxidants.

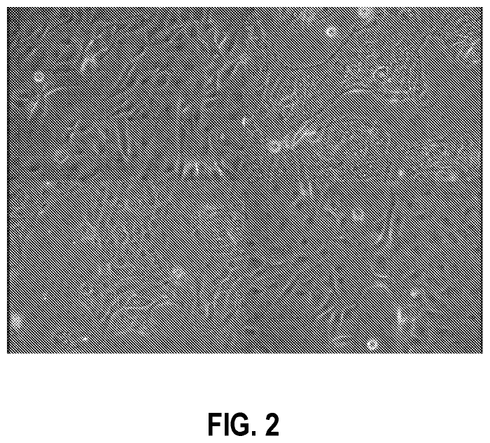

Relative levels of ROS production were then determined by the relative fluorescent intensity at Ex/Em=485/535 nm using Abcam DCFDA cellular ROS assay kit. At 6 hours after infusion, the relative ROS production in NS and LR were 4.9 and 6.6-fold higher than control, vs 1.8-fold with AL (P<0.001). At 4 hours after infusion, the relative ROS production in NS and LR were 3.0-fold and 4.9 fold higher than control, vs a 1.5-fold increase with AL. The ROS increase in AL could be mitigated with the addition of antioxidants. Ascorbic acid and ulin-A-statin, added to AL at physiologic concentrations, led to the best ROS mitigation, with a 0.7-fold ROS expression at 4 hours compared with control and 0.6-fold expression at 6 hours relative to control, (P<0.001 for both) (FIG. 3). The reduction in ROS production was greater with ascorbic acid than with ulin-A-statin, because AL+ascorbic acid alone led to a 0.7-fold expression of ROS relative to control at 4 and 6 hours (P<0.001); whereas AL+UTI led to similar ROS production to AL alone (1.3 and 1.7 fold increase relative to control at 4 and 6 hours respectively), The 1.3 and 1.7 fold increases are reflected accurately in FIG. 3A. Findings were confirmed with DCFDA staining visualized under immunofluorescence microscopy (FITC, 20×100 ms exposure) (FIG. 3). Due to improvements in the ROS pathway with the addition of ascorbic acid and UTI, tissue explant testing was performed with the various potential components of AL (AL+ascorbic acid, AL+UTI, AL+both) and the lowest cell death rate was noted in AL+both (FIG. 3).

### Up-Regulation of Apoptotic Pathways in Cells Exposed to Normal Saline

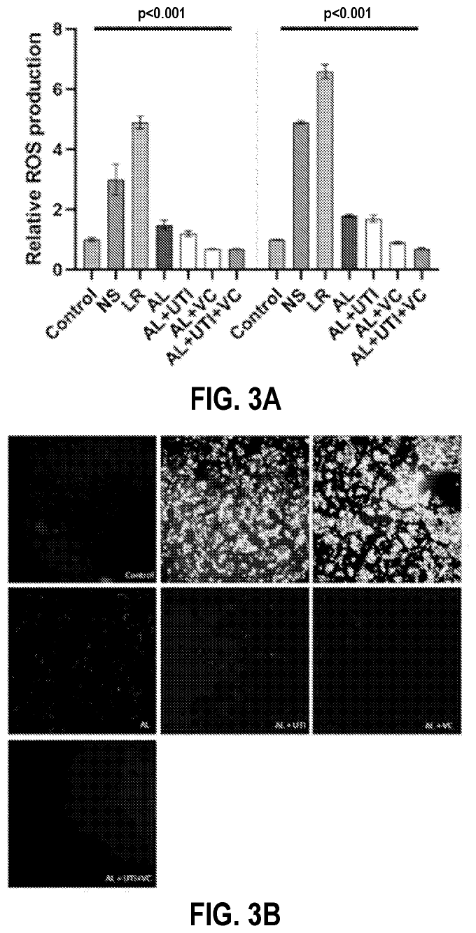

Given the higher apoptosis in NS, but higher ROS production in LR, common pathways of apoptosis were evaluated between control, NS, and various formulations of AL to identify other pathways of cell death with the amnioinfusion. Caspase 3, a final executioner of cell death, was upregulated in NS compared to control and AL+UTI (P=0.041). Cyclin-dependent kinase inhibitor 1 (P21) was upregulated in NS compared with control and all formulations of AL (P=0.006). BCL2, a known inhibitor of apoptosis, as well as the BCL2-associated X-protein (BAX), a known member of mitochondrial-induced cell death pathways, were quantified and the relative BCL20-to-BAX ratio was significantly decreased in the NS group compared to control (P=0.0121). The highest BCL2-to-BAX ratio was in the AL+ulin-A-statin (FIG. 4). Given the benefit from a ROS-production perspective with ascorbic acid and an improvement in the reduction of BCL2 pathway cell death with ulin-A-statin, the final formulation of AL was comprised of, in order of decreasing concentration: sodium chloride, sodium gluconate, sodium acetate, albumin, potassium chloride, d-glucose, magnesium chloride, ascorbic acid, ulin-A-statin, and zinc chloride.

### DISCUSSION

In this study, NS and LR exposure to human amniotic membranes in vitro, both in cell culture and in tissue explants, led to cell death. This damage persisted because cells had higher levels of apoptosis days after exposure (compared to immediately after exposure), and raises concerns that these fluids may not adequately support the amniotic membranes during fetal procedures. The principal mechanism of cell injury in culture was ROS-mediated, an important finding given how heavily ROS is implicated in the pathogenesis of PPROM. However, given the higher ROS production in LR compared to NS, all apoptotic pathways were evaluated, and alterations in the p21 and BAX/BCL2 pathways were also noted with NS.

Although there is evidence that the uterine defect size at the time of fetal interventions is associated with PPROM after surgery, this does not fully explain postoperative PPROM rates. An example of this is the increased rates of PPROM after fetoscopic myelomeningocele (MMC) closure vs open MMC closure, where higher rates of PPROM are seen even though the fetoscopic incisions are much smaller, but almost all the amniotic fluid is replaced by warmed LR. Habli et al presented a PPROM rate of 61% in the setting of serial transabdominal amnioinfusions through a small single 6F port. Conversely, the PPROM rate after fetoscopic laser through a significantly larger single 10F or even 12F port is approximately 33%. A difference between these 2 situations is the frequent (at least once per week) infusion of NS or LR into the amniotic cavity in the setting of serial amnioinfusions, something not done in fetoscopic laser surgery.

Apoptosis at the level of the amniotic epithelium has been shown in vivo to precede rupture of membranes and increased apoptosis has been associated with increased membrane fragility. It is therefore biologically plausible that the amnioinfusion with NS and LR at the time of fetal intervention could increase membrane fragility, predisposing the membrane to be more prone to PPROM. This is further supported by the increased ROS production with amnioinfusions, which is an established pathway for PPROM. Some may argue that given the normal turnover of amniotic fluid, exposing the amnion to LR or NS for a brief period should not impact the membrane; however, in vitro, Applicant found the damage to be lasting, with increasing cell death even after the cells spend substantial time back in culture media.

### Clinical Implications

of complicated gastroschisis without success; however, these were undertaken with LR. Evaluation of a more amniotic fluid-like fluid with added antioxidants to better protect the exposed bowel from meconium could prove different. Furthermore, if this fluid proves to be safe and effective for the amniotic membranes without increased risk of PPROM and preterm birth, AL may provide an opportunity for use in amniotic fluid restoration in other disease states still.

### CONCLUSION

NS and LR cause significant cell damage and death to human amniotic epithelium, both in cell and in organotypic cultures in vitro. Our novel synthetic amniotic fluid, Amnio-well, when exposed to late third-trimester amniotic membranes, does not cause amnion cell death or damage in vitro.

TABLE
Composition of commonly used fluids for amnioinfusions compared with
amniotic fluid and Amnio-well. The final composition of Amnio-well, in order of decreasing
concentration, diluted into 1 L of sterile water is sodium chloride, sodium gluconate, sodium
acetate, albumin, potassium chloride, d-glucose, magnesium chloride, ascorbic acid, urinary
trypsin inhibitor, and zinc chloride. At the conclusion of composition, the pH is checked, and
sodium hydroxide is added as needed to confirm a pH of 7.3 to 7.5. Forde.
A novel synthetic amniotic fluid. Am J Obstet Gynecol MFM 2023.

| | | Na | Cl | K | Ca | Mg | Albumin | Glucose |
|---|---|---|---|---|---|---|---|---|
| Variable | pH | (mEq/L) | (mEq/L) | (mmol/L) | (mg/dL) | (mg/dL) | (mg/dL) | (mg/mL) |
| Normal | 5-5.5 | 154 | 154 | — | — | — | — | — |
| saline | | | | | | | | |
| solution | | | | | | | | |
| Lactated | 6.5 | 130 | 109 | 4 | 2.7 | — | — | — |
| Ringer's | | | | | | | | |
| solution | | | | | | | | |
| Second- | 7.4 | 135 | 105 | 4.5 | 6 | 1.7 | 0.3 | 30 |
| trimester | | | | | | | | |
| amniotic | | | | | | | | |
| fluid | | | | | | | | |
| Amnio- | 7.4 | 135 | 105 | 4.5 | 6 | 3 | 0.3 | 30 |
| well | | | | | | | | |

### Example: Experiments Demonstrating UTI and Vitamin C Synergy

P21 (also referred to as CDKNA1) is a key player in cell death cycles. P21 is upregulated in Normal Saline relative to control in Applicant's amnioinfusion model. However, P21 is not upregulated in the presence of Amnio-well having both UTI and ascorbic acid.

Amino-well+UTI and or ascorbic acid (Vitamin C) was tested at three different concentrations of both UTI and/or ascorbic acid, as shown in the following table.

TABLE
Tested compositions.

| Groups | Designation | Per 1 L | |
|---|---|---|---|
| Control | C | | |
| Normal Saline | NS | | |
| Amnio-well + Vitamin C | VC1 | 400 | mg |
| Amnio-well + Vitamin C | VC2 | 800 | mg |
| Amnio-well + Vitamin C | VC3 | 2000 | mg |
| Amnio-well + UTI | UTI1 | 37.5 | mg |
| Amnio-well + UTI | UTI2 | 75 | mg |
| Amnio-well + UTI | UTI3 | 187.5 | mg |

| | | |
|---|---|---|
| Amnio-well + Vitamin C + UTI | B1 | 400 mg + 37.5 mg |
| Amnio-well + Vitamin C + UTI | B2 | 800 mg + 75 mg |
| Amnio-well + Vitamin C + UTI | B3 | 2000 + 187.5 |

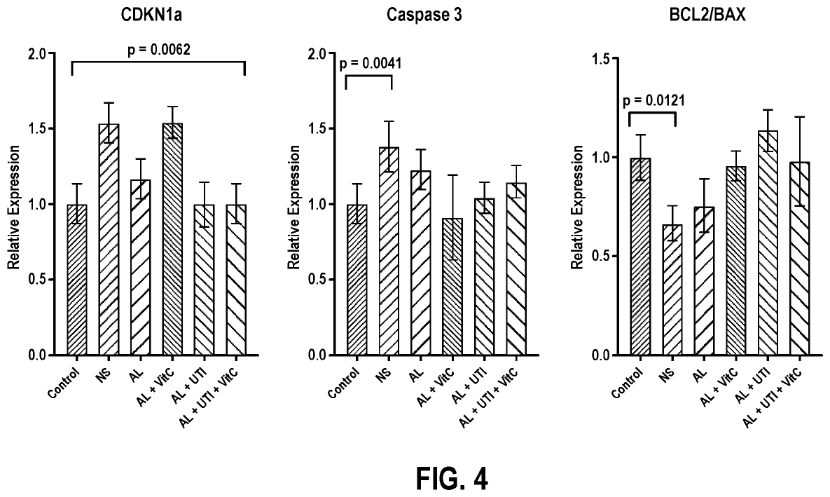

Applicant found that P21 is upregulated in Normal Saline compared with Control. P21 was downregulated in Formulation VC1 and Formulation VC2, (containing 400 mg and 800 mg of Vitamin C, respectively) but significantly upregulated in Formulation VC3 containing the highest level (2000 mg) of Vitamin C per liter. Formulation UTI1 (having 37.5 mg/L of UTI, but no ascorbic acid) was observed to have P21 mRNA levels equivalent to control and downregulated in B1, B2, and B3 (having both Vitamin C and UTI, at increasing level). Interestingly, Formulation B3, having both actives, (2000 of Vitamin C and 187.5 mg of UTI), showed downregulation of P21. Thus, UTI regulated the higher Vitamin C concentration. (FIG. 5.)

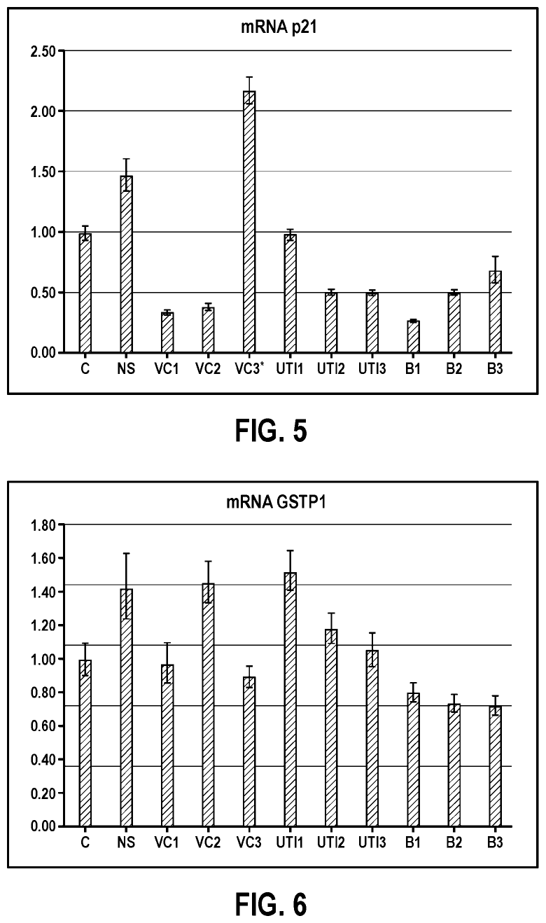

With regard to reactive O2 species, based on RNA sequencing, upregulation in many oxidative stress pathways had been observed. One marker that can be measured using PCR is Glutathione S-Transferase Pi 1 (GSTP1). GSTP1 is upregulated in Normal Saline (as expected), however, GSTP1 is only consistently downregulated with the combination of both vitamin C and UTI, as shown in FIG. 6. Formulations VC1, VC2, VC3, UTI1, UTI2, and UTI3 did not cause a decrease in GSTP1 expression, however, the synergy of both UTI and vitamin C, even at the lowest levels of each (as shown with the effects of Formulation B1), was found to lead to a significant decrease in GSTP1. Thus, the combination of both vitamin C and UTI yielded unexpected results in downregulating GSTP1, a marker of oxidative stress in tissue.

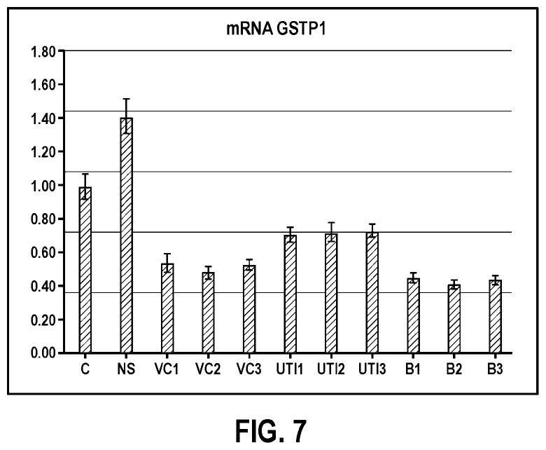

Lastly, BAX (Bcl-2-associated X protein), a protein that regulates apoptosis, is observed to be elevated when contacted with Normal Saline as compared to a control, was found to be decreased as compared to NS and control across the board with all the concentrations of UTI, VC and combinations. FIG. 7.

### REFERENCES

Salomon L J, Sotiriadis A, Wulff C B, Odibo A, Akolekar R. Risk of miscarriage following amniocentesis or chorionic villus sampling: systematic review of literature and updated meta-analysis. Ultrasound Obstet Gynecol 2019; 54:442-51.
Kabagambe S K, Jensen G W, Chen Y J, Vanover M A, Farmer D L. Fetal surgery for myelomeningocele: a systematic review and meta-analysis of outcomes in fetoscopic versus open repair. Fetal Diagn Ther 2018; 43:161-74.
Forde B, Habli M. Unique considerations: preterm prelabor rupture of membranes in the setting of fetal surgery and higher order pregnancies. Obstet Gynecol Clin North Am 2020; 47:653-69.
Devlieger R, Millar L K, Bryant-Greenwood G, Lewi L, Deprest J A. Fetal membrane healing after spontaneous and iatrogenic membrane rupture: a review of current evidence. Am J Obstet Gynecol 2006; 195:1512-20.
Bourne G. The foetal membranes. A review of the anatomy of normal amnion and chorion and some aspects of their function. Postgrad Med J 1962; 38:193-201.
Papanna R, Mann L K, Moise Jr K J, et al. Histologic changes of the fetal membranes after fetoscopic laser surgery for twin-twin transfusion syndrome. Pediatr Res 2015; 78:247-55.
Snowise S, Mann L K, Moise Jr K J, Johnson A, Bebbington M W, Papanna R. Preterm prelabor rupture of membranes after fetoscopic laser surgery for twin-twin transfusion syndrome. Ultrasound Obstet Gynecol 2017; 49:607-11.
Chmait R H, Kontopoulos E V, Chon A H, Korst L M, Llanes A, Quintero R A. Amniopatch treatment of iatrogenic preterm premature rupture of membranes (iPPROM) after fetoscopic laser surgery for twin-twin transfusion syndrome. J Matern Fetal Neonatal Med 2017; 30:1349-54.
Sodium Chloride Injection-FDA Prescribing Information, Side Effects, and Uses. Available at: www.drugs.com/pro/sodium-chloride-injection.html. Accessed May 1, 2021.
Ringer's Lactate Injection-FDA Prescribing Information, Side Effects, and Uses. Available at: www.drugs.com/pro/lactated-ringers.html. Accessed May 1, 2021.
Motedayyen H, Esmaeil N, Tajik N, et al. Method and key points for isolation of human amniotic epithelial cells with high yield, viability and purity. BMC Res Notes 2017; 10:552.
Modena A B, Fieni S. Amniotic fluid dynamics. Acta Biomed 2004; 75 (Suppl1): 11-3.
ThermoFisher Scientific. Fixable Viability Dye eFluor® 780 product information. 2023. Available at: www.thermofisher.com/order/catalog/product/65-0865-14. Accessed Jul. 1, 2021.
Apotracker Green product information. 2021. Available at: www.biolegend.com/en-us/products/apotracker-green-1 8527? GroupID=GROUP22. Accessed Jul. 1, 2021.
Invitrogen. LIVE/DEAD™ Viability/cytotoxicity Kit, for mammalian cells product information. 2023. Available at: www.thermofisher.com/order/catalog/product/L3224 #/L3224. Accessed Jun. 28, 2021.
Mercer B M, Abdelrahim A, Moore R M, et al. The impact of vitamin C supplementation in pregnancy and in vitro upon fetal membrane strength and remodeling. Reprod Sci 2010; 17:685-95.
Casanueva E, Ripoll C, Tolentino M, et al. Vitamin C supplementation to prevent premature rupture of the chorioamniotic membranes: a randomized trial. Am J Clin Nutr 2005; 81:859-63.
U S Food and Drug Administration. Vitamin C dosing guide. 2017. Available at: www.accessdata.fda.gov/drugsatfda_docs/label/2017/209112s000lbl.pdf. Accessed Jul. 1, 2021.
Kaga N, Katsuki Y, Futamura Y, Obata M, Shibutani Y. Role of urinary trypsin inhibitor in the maintenance of pregnancy in mice. Obstet Gynecol 1996; 88:872-82.
Kobayashi H, Suzuki K, Sugino D, Terao T. Urinary trypsin inhibitor levels in amniotic fluid of normal human pregnancy: decreased levels observed at parturition. Am J Obstet Gynecol 1999; 180:141-7.
Hayashi M, Oya A, Miyake H, Nakai A, Takeshita T. Effect of urinary trypsin inhibitor on preterm labor with high granulocyte elastase concentration in cervical secretions. J Nippon Med Sch 2010; 77:80-5.
Matsuda Y, Yunohara N. Effects of urinary trypsin inhibitor in patients at risk for premature labor with a bulging fetal membrane. Fetal Diagn Ther 2002; 17:69-74.
abcam. DCFDA/H2DCFDA-cellular ROS Assay Kit (ab113851) general product information. Available at: www.abcam.com/dcfda-h2dcfda-cellular-ros-assay-kit-ab113851.html. Accessed Jul. 1, 2021.
Habli M, Riddle S, Null G, et al. Outcome of Pregnancies Complicated by Fetal Renal Failure (FRF) Treated by Percutaneous Needle amnioinfusions versus Amnioport. Am J Obstet Gynecol 2022; 226: S49-50.
Kumar D, Moore R M, Mercer B M, Mansour J M, Redline R W, Moore J J. The physiology of fetal membrane weakening and rupture: insights gained from the determination of physical properties revisited. Placenta 2016; 42:59-73.
McLaren J, Taylor D J, Bell S C. Increased incidence of apoptosis in non-labour-affected cytotrophoblast cells in term fetal membranes overlying the cervix. Hum Reprod 1999; 14:2895-900.
Kumagai K, Otsuki Y, Ito Y, Shibata M A, Abe H, Ueki M. Apoptosis in the normal human amnion at term, independent of Bcl-2 regulation and onset of labour. Mol Hum Reprod 2001; 7:681-9.
Woods Jr. J R. Reactive oxygen species and preterm premature rupture of membranes-a review. Placenta 2001; 22 (SupplA): S38-44.
Bryant-Greenwood G D, Yamamoto S Y. Control of peripartal collagenolysis in the human chorion-decidua. Am J Obstet Gynecol 1995; 172:63-70.
Richardson L S, Vargas G, Brown T, et al. Discovery and characterization of human amniochorionic membrane microfractures. Am J Pathol 2017; 187:2821-30.
Wooding P, Burton G. Comparative placentation. Structures, functions and evolution. Berlin, Germany: Springer; 2008.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A synthetic amniotic fluid composition comprising ulin-A-statin/urinary trypsin inhibitor (UTI) and ascorbic acid.

2. The synthetic amniotic fluid composition of claim 1 comprising from about 37.5 mg/L to about 75 mg/L UTI and from about 400 mg/L to about 800 mg/L ascorbic acid.

3. The synthetic amniotic fluid composition of claim 1, comprising UTI and ascorbic acid at a ratio of about 1:10.

4.-12. (canceled)

13. The synthetic amniotic fluid composition of claim 1, further comprising one or more of sodium chloride, sodium gluconate, albumin, potassium chloride, d-glucose, magnesium chloride, zinc chloride, and combinations thereof.

14.-19. (canceled)

20. The synthetic amniotic fluid composition of claim 1, wherein said composition comprises about 135 mEq/L sodium, about 4.5 mmol/L potassium, about 6 mg/dL calcium, about 2 mg/dL magnesium, about 0.3 mg/dL albumin, and about 30 mg/dL glucose.

21.-34. (canceled)

35. A method for reducing the likelihood of a premature delivery, comprising administering a synthetic amniotic fluid composition comprising ulin-A-statin/urinary trypsin inhibitor (UTI) and ascorbic acid to an individual in need thereof.

36. The method of claim 35 wherein said individual is at risk of postoperative PPROM.

37. The method of claim 35 wherein said individual has undergone one or both of fetoscopic myelomeningocele (MMC) closure and open MMC closure.

38. (canceled)

39. The method of claim 35, wherein said administering comprises introducing said composition via transabdominal infusion and/or amnioinfusion.

40. The method of claim 35, wherein said administering comprises introducing said composition via an amnioinfusion at least one time per week.

41. (canceled)

42. The method of claim 35, wherein said administering is carried out during a fetal intervention.

43. A method for improving viability of an amniotic epithelial cell, comprising contacting said amniotic epithelial cell with a synthetic amniotic fluid composition comprising ulin-A-statin/urinary trypsin inhibitor (UTI) and ascorbic acid.

44. (canceled)

45. The method of claim 43 wherein said improving viability comprises one or more of reduced cellular shrinkage, reduced nuclear fading, and reduced membrane blebbing.

46. (canceled)

47. (canceled)

48. (canceled)

49. The method of claim 43, wherein said amnion epithelial cell is in cell culture or amniotic tissue.

50. (canceled)

51. The method of claim 43, wherein said contacting is ex vivo or in vivo.

52. (canceled)

53. (canceled)

54. (canceled)

55. The method of claim 43, wherein said contacting comprises replacing or supplementing a portion of an amniotic fluid in an individual in need thereof.

56. A method for restoring and/or improving amniotic fluid volume in an individual in need thereof, comprising administering a synthetic amniotic fluid composition comprising ulin-A-statin/urinary trypsin inhibitor (UTI) and ascorbic acid to the amniotic sac of said individual.

57.-62. (canceled)

63. The method of claim 35 wherein the synthetic amniotic fluid composition comprises from about 37.5 mg/L to about 75 mg/L UTI and from about 400 mg/L to about 800 mg/L ascorbic acid.

64. The method of claim 43 wherein the synthetic amniotic fluid composition comprises from about 37.5 mg/L to about 75 mg/L UTI and from about 400 mg/L to about 800 mg/L ascorbic acid.

65. The method of claim 56 wherein the synthetic amniotic fluid composition comprises from about 37.5 mg/L to about 75 mg/L UTI and from about 400 mg/L to about 800 mg/L ascorbic acid.
